(19) United States
(12) Patent Application Publication
(10) Pub. No.: US 2024/0053356 A1
(43) Pub. Date: Feb. 15, 2024

(54) AFFINITY SELECTION BY MASS SPECTROMETRY WORKFLOW USING MAGNETIC PARTICLES

(71) Applicant: DH Technologies Development Pte. Ltd., Singapore (SG)

(72) Inventor: Chang LIU, Richmond Hill (CA)

(73) Assignee: DH Technologies Development Pte. Ltd., Singapore (SG)

(21) Appl. No.: 18/264,613

(22) PCT Filed: Feb. 10, 2022
(86) PCT No.: PCT/IB2022/051214

§ 371 Date: Aug. 8, 2023

Related U.S. Application Data
(60) Provisional application No. 63/147,827, filed on Feb. 10, 2021.

(51) Int. Cl. G01N 33/68; G01N 33/543; G01N 33/574

(52) U.S. Cl. CPC G01N 33/6848; G01N 33/54326; G01N 33/54346; G01N 33/57488

(57) ABSTRACT

Methods and systems for conducting affinity selection by mass spectrometry in high throughput assays are disclosed herein. The methods can comprise identifying a set of hit com-pounds having a selected affinity to a binding target immobilized onto a magnetic particle. Methods can comprise forming an assay mixture within an assay vessel comprising a plurality of drug candidates and a binding target immobilized onto a magnetic particle. Methods also can comprise preparing at least a portion of the assay mixture for mass analysis transferring a sample containing the set of hit compounds to an open port sampling interface of a mass spectrometer.

### CROSS-REFERENCE TO RELATED APPLICATION

This application is being filed on Feb. 10, 2022, as a PCT Patent International Application and claims the benefit of and priority to U.S. Provisional Patent Application No. 63/147,827, filed on Feb. 10, 2021, which application is hereby incorporated herein by reference.

### BACKGROUND

Affinity selection by mass spectrometry (ASMS) involves the binding of candidate molecules to immobilized or soluble receptors and has been used for screening large compound libraries in a time and cost-effective manner. The conventional ASMS workflow is based on solution phase incubation, wherein a binding target is added to a mixture of the drug molecules. After incubating the binding target with the mixture of drug molecules, unbound drug molecules can be washed from the mixture leaving only those compounds having affinity for the target protein as a drug-protein complex. This selection-by-affinity can be followed by elution of the bound compounds from the target protein during a preparatory workup prior to identification by mass spectrometry.

Subsequent chromatographic methods also have been employed to separate the eluted drug molecules from the target protein and other ligand binding assay components. Ultrafiltration, spin-column, and size-exclusion chromatography have conventionally been employed to isolate hit compounds bound to the target protein from unbound compounds and other components within the assay mixture. Such chromatography steps often have been employed as the first chromatography step of two dimensional chromatographical methods, where the second dimension constitutes separation of the bound hit compound from the target protein prior to mass spectral analysis.

Multi-dimensional chromatography operations typically must be performed in a serial manner. In the context of high throughput screening methods, where hundreds or thousands of samples are analyzed within a given well plate, time-consuming preparatory and analytical procedures can greatly extend the time required to complete the screening, or alternatively limit the number of compounds that can be screened. Methods able to avoid such bottlenecks during preparatory and analytical stages of high throughput screening are desired.

### SUMMARY

This summary is provided to introduce a selection of concepts in a simplified form that are further described below in the detailed description. This summary is not intended to identify required or essential features of the claimed subject matter. Nor is this summary intended to be used to limit the scope of the claimed subject matter.

The technologies described herein are directed to systems and methods for efficiently screening the binding affinity of compounds within a large compound library. During such processes, compounds may be separated from an assay mixture based on a selected affinity to a binding target and subsequently identified by mass spectrometry. The systems and methods may be ASMS methods compatible with automated high throughput screening of compounds for affinity to a binding target, and may exclude both preparatory and analytical chromatography operations. As chromatography operations typically must be performed in a serial manner with respect to analysis of a plurality of assay mixtures, such as in a high-density microtiter plate, the presently disclosed systems and methods provide significant benefits to the capacity of HTS processes. The methods disclosed herein may incorporate direct sampling of the mixtures resulting from individual screening assays for mass spectrometry analysis.

Disclosed herein are methods for identifying a set of hit compounds having a selected affinity to a binding target from a plurality of drug candidates. Generally, methods disclosed herein can comprise forming an assay mixture within an assay vessel, the assay mixture comprising a plurality of drug candidates and a binding target immobilized onto a magnetic particle, preparing at least a portion of the assay mixture for mass analysis, and transferring a sample containing the set of hit compounds to an open port sampling interface of a mass spectrometer. In certain aspects, transferring the set of hit compounds can occur prior to elution of the hit compounds from the binding target. Alternatively, the set of hit compounds may be eluted from the binding target before transferring to an open port sampling interface. Methods disclosed herein may be particularly suited for automated and high throughput applications where identifying compounds having a selected affinity for a particular binding target can be done at large scale with minimal intervention. Methods disclosed herein can comprise the operations above generally in any order, partially or completely, as demonstrated by the several embodiments and examples provided herein.

In certain aspects, forming the assay mixture can comprise introducing the plurality of drug candidates into the assay vessel by serially adding individual compounds from a compound library to the assay vessel; introducing magnetic particles into the assay vessel; and, optionally, incubating the plurality of drug candidates and the magnetic particles under assay conditions. The magnetic particles including binding sites operative to bind with one or more target compounds.

In certain aspects, preparing at least a portion of the assay mixture can comprise separating one or more components of the assay mixture from the set of hit compounds, i.e. separating unbound compounds from the bound compound-magnetic particle components, and separating hit compounds from the binding targets on the magnetic particles.

In certain aspects, transferring the sample can be conducted partially or completely prior to preparing the assay mixture for mass analysis.

In certain aspects, the sample containing the set of hit compounds comprises the magnetic particles in the form of a bound compound-magnetic particle component within the assay mixture.

Certain aspects can further comprise trapping the magnetic particles within a carrier flow of the mass spectrometer by selectively activating a magnetic force, and subsequently releasing the magnetic particles to a waste flow by selectively deactivating the magnetic force. Such aspects can comprise trapping the magnetic particles within the open port sampling interface or within a downstream component of the open port sampling interface prior to directing the carrier flow to an ion source for ionization.

In certain aspects, the magnetic particles can be discarded via exhaust from a sample vaporization chamber of the mass spectrometer that includes a trap to isolate the magnetic particles from the inlet of the mass spectrometer.

In certain aspects, transferring a sample containing the set of hit compounds can comprise transferring a sample of the assay mixture directly from the assay vessel to the open port sampling interface, trapping bound compound-magnetic particle components in a magnetic trap in fluid communication with the open port sampling interface, and switching a capture liquid flowing through the trap to a solvent operative to release the bound compounds from the binding sites on the magnetic particles.

Preparing the assay mixture can consist of inserting a magnet into the assay mixture to retain the magnetic particles adjacent to the magnet; removing the magnet and retained magnetic particles from the assay mixture; optionally washing the magnetic particles retained adjacent to the magnet with a wash solution; and contacting the magnetic particles with an eluent, optionally while the magnetic particles are retained adjacent to the magnet, thereby separating hit compounds from the binding target(s) on the magnetic particle, transferring the eluted hit compounds to the open port sampling interface.

Alternatively, preparing the assay mixture can consist of applying a magnetic force adjacent the assay vessel to retain the magnetic particles within the assay vessel; aspirating at least a portion of the assay mixture from the assay vessel; optionally washing the magnetic particles within the assay vessel; adding an eluent to the assay vessel to elute hit compounds from the binding target, and transferring the hit compounds from the assay vessel into an open port sampling interface. In some aspects, the transferring may comprise transferring the magnetic particles and the hit compounds from the assay vessel into the open port sampling interface. In these aspects, a magnetic trap is located to trap and isolate magnetic particles from the hit compounds before the hit compounds are ionized and drawn into an inlet of a mass spectrometer. In other aspects, the magnetic particles may be isolated within the assay vessel, using an applied magnetic force, and the hit compounds are separated from the magnetic particles by ejecting the hit compounds from the assay vessel into the open port sampling interface.

In certain aspects, transferring the sample containing the set of hit compounds to the open port sampling interface can comprise acoustic ejection.

Certain aspects can further comprise analyzing the set of hit compounds by mass spectrometry, without liquid chromatography.

Automated, high throughput screening (HTS) systems are also disclosed herein, and generally in accord with the methods disclosed above. HTS systems disclosed herein can comprise, in certain embodiments, a assay vessel preparation module configured to introduce a plurality of compounds from a compound library into an assay vessel, an assay module configured to conduct a binding assay comprising introducing magnetic particles into the assay vessel, the magnetic particles including at least one binding site for binding with at least one target compound, and an analysis module configured to transfer sample from the assay vessel for analysis. In embodiments where the assay vessel comprises a sample well of a microtiter well plate, the analysis module may be operative to transfer a sample from each well of the well plate into an open port sampling interface of a mass spectrometer and conduct a mass analysis of each sample.

Sample preparation information associated with each assay vessel is generated to correspond to the compounds introduced to that well as well as any other relevant sample preparation information, such as magnetic particle binding targets, sample preparation methods, incubation time, etc. In some aspects an identifier, such as a barcode, may be associated with sample well plate that includes a plurality of sample wells (assay vessels). Some or all of the modules may include a bar code reader to identify the sample well plate and be operative to locate each sample well within the sample well plate and associate that sample well with the corresponding sample preparation information associated with that sample well.

The sample preparation information may be used by the analysis module to correlate the mass analysis results generated for each sample well with the sample information associated with that sample well. The correlation may identify which compounds appear in each mass spectra from the mass analysis results (i.e. the hit compounds) based on the associated sample well information and sample information for that sample well. The sample information may include, for instance, identifying information corresponding to the one or more compound(s) introduced into that assay vessel, is associated with the sample well. The sample information may include, for instance, identifier(s) indicative of each of the one or more compounds, reagents, or other information related to analysis of the sample well.

Accordingly, the system is operative to identify which compound set was introduced into a particular sample well and which compound(s), i.e. bound compounds, were identified by the mass analysis.

In some embodiments, an automated high throughput screening system is provided. The system comprising: an assay vessel preparation module configured to introduce a plurality of compounds from a compound library into an assay vessel; an assay module configured to conduct a binding assay comprising the plurality of compounds and magnetic particles including at least one binding site for binding with at least one target compound in the assay vessel; and an analysis module configured to transfer sample from the assay vessel into an open port sampling interface for capture and transfer to a mass spectrometer that conducts a mass analysis on the transferred sample. In some embodiments, the assay vessel comprises a sample well of a microtiter well plate and wherein the assay vessel preparation module is operative to selectively introduce compounds into each sample well of the well plate. In these embodiments, a sample identifier may typically be provided for each well plate and each sample well is identified based on the well plate identifier and a location, or coordinate position, of that sample well on the well plate. In some embodiments, the assay vessel may comprise an aliquot tube or vial and wherein the assay vessel preparation module is operative to selectively introduce compounds into each aliquot tube or vial. In these embodiments sample identifiers may typically be provided on each aliquot tube or vial. The sample identifiers may be in the form, for instance, of a vessel identifier that is correlated to the sample information associated with that assay vessel.

In some embodiments the analysis module may include an acoustic droplet ejector operative to eject sample with an assay vessel into an open port interface for transfer to an ionization source for ionization. The acoustic droplet ejector may be configured, for instance, to transfer sample from multiple assay vessels at a rate of about 1 Hz.

In some aspects, the assay vessel preparation module is configured to associate the identifier with an identity of each compound introduced into that assay vessel. In some aspects, the assay vessel preparation module receives a list of one or more compounds to introduce to an assay vessel and either associates an identifier for that assay vessel or receives an identifier of a corresponding assay vessel to be used to receive the listed one or more compounds.

In some embodiments, the analysis module is configured to correlate the mass analysis results generated from sample transferred from an assay vessel with the sample information associated with that sample well.

It is contemplated that each module of the system can be operated independently to achieve a particular end point, but also in communication with other modules such that each can be universally adapted for use with any number of other modules.

In certain aspects, the assay vessel preparation module can comprise an automated liquid dispenser. In certain aspects, the assay vessel preparation module can comprise an acoustic dispenser.

In certain aspects, the magnetic binding particle can comprise a magnetic bead, the magnetic bead comprising a streptavidin-biotin complex with a protein binding target.

In certain aspects, the analysis module can comprise an acoustic droplet ejector. Such aspects can be configured to transfer at least 1 sample per second from the well plate to the open port sampling interface.

Also disclosed herein are open port sampling interfaces for a mass spectrometer. Open port sampling interfaces generally can comprise an inner channel in fluid connection with an ionization chamber of the mass spectrometer, and an outer channel in fluid connection with a solvent source, the inner and outer channels defining a solvent flow path from the solvent source to the ionization chamber; an open port positioned near a junction between the outer channel and inner channel; an electromagnetic trap positioned within the solvent flow path and downstream of the open port, the electromagnetic trap configured to selectively retain magnetic particles entering the inner channel at the open port in an operative state; and a solvent flow path diverter positioned downstream of the electromagnetic trap and configured to selectively divert the solvent flow from an analytical flow path to a waste flow path when the electromagnetic trap is in a non-operative state.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects and embodiments may be directed to various feature combinations and sub-combinations described in the detailed description.

### BRIEF DESCRIPTION OF THE DRAWINGS

FIG. 1 shows the MagMASS method of using magnetic particles to capture drug molecules with protein binding affinity.

FIG. 2 is a schematic representation of an open port sampling interface (OPI) used in embodiments.

FIG. 3 depicts an embodiment of a method for identifying and separating compounds based on a selected affinity.

FIG. 4 depicts a method for identifying and separating compounds based on a selected affinity according to an embodiment.

FIG. 5 depicts a possible system for implementing the method of FIG. 4.

FIG. 6 depicts a method for identifying and separating compounds based on a selected affinity according to a further embodiment.

FIG. 7 depicts a possible system for implementing the method of FIG. 6.

FIG. 8 depicts a method for identifying and separating compounds based on a selected affinity according to an additional embodiment.

FIG. 9 depicts a possible system for implementing the method of FIG. 8.

FIG. 10 depicts a possible variation of the system of FIG. 9.

FIG. 11 depicts a method for identifying and separating compounds based on a selected affinity.

### DETAILED DESCRIPTION

As generally stated above, methods disclosed herein can comprise forming an assay mixture within an assay vessel, where the assay vessel comprises a plurality of drug candidates and a magnetic binding particle. Compounds available to make up the plurality of drug candidates generally can be any that are soluble and stable under the conditions of the binding assay, and are not limited to any particular structure, type, source, or class of compounds.

In certain aspects, the plurality of drug candidates can be derived from any source compatible with the affinity assay. In certain aspects the plurality of drug candidates can be individually and serially selected from a large synthetic library of compounds such as small molecules. Small molecules often can be stored as a highly concentrated solution in organic solvents (e.g., dimethyl sulfoxide (DMSO)) as intended for dilution within the binding assay buffer. Biological compounds such as peptides, nucleic acids, lipids, and the like are also contemplated as drug candidates to binding targets suitable for the screening methods disclosed herein.

Physical properties of compounds, other than their potential affinity to a binding target, do not necessarily restrict any particular compound from applicable to the disclosed methods. In certain aspects, the compounds having some solubility in aqueous solutions can prove advantageous according to their compatibility with common assay buffers and screening assay conditions. In such aspects, the introduction compounds into may be added as a small volume of highly concentrated solution in an organic solvent such as DMSO. Liquid dispensers (e.g., Mosquito® dispensers, available from SPT Labtech Ltd.) are known in the art for this purpose, and can quickly and precisely provide nanoliter scale amounts of compound solutions to an assay vessel to be later diluted by assay buffer. Acoustic dispensing methods (e.g., Echo® acoustic dispensers, available from Labcyte Inc.) are also known in the art to achieve a similar result. Of course, compounds also may be transferred by any mechanism known in the art and suitable for the purpose of the affinity selection assay.

In certain aspects, drug candidates may be derived from natural or synthetic sources. Indeed, drug candidates may be selected as a sample of a single natural product or extract thereof. For instance, maceration of a naturally occurring species in the presence of a solvent can provide a complex compound mixture that can be added directly to the assay vessel in microliter amounts. Such aspects may also comprise physical separations and reductions to ensure that a sufficient amount of trace natural products present in the mixture are adequately represented.

In certain aspects the assay mixture can comprise a plurality of compounds in a range from 20 to 20,000, from 50 to 10,000, from 100 to 5,000, from 250 to 4,000, or from 400 to 2,500. In other aspects, the assay mixture can comprise a number of unique compounds in a range from 10 to 1,000, from 25 to 800, from 50 to 500, or from 100 to 250. Alternative methods, concentrations, compound combinations, and apparatus suitable to prepare a selectivity assay mixture are also contemplated herein.

Separately, forming the assay mixture can comprise introducing a magnetic particle to the assay vessel. As used in reference to terms herein, for instance, as applied immediately above to “a magnetic particle,” the terms “a,” “an,” and “the” are intended to include plural alternatives, e.g., at least one. For instance, the disclosure of “a magnetic particle” is meant to encompass one magnetic particle, or mixtures or combinations of more than one magnetic particle, unless otherwise specified. Magnetic particles contemplated herein are not particularly limited by their size, shape or composition. Therefore, in certain aspects, “a magnetic particle” can comprise any amount of a nanoparticulate ferromagnetic particle, a magnetic bead comprising a magnetic core and polymeric coating, and combinations thereof.

In certain aspects, the magnetic particles can each comprise one or more binding targets immobilized on a surface of the particle accessible to the compounds in the assay mixture. The binding target can be covalently or noncovalently immobilized on the magnetic particle. The binding target can be any class of material or structure where determining a selected affinity to the binding target is desirable. In certain aspects, the binding target can be a protein such as an antibody or antigen, a protein fragment, a nucleic acid, a nucleic acid fragment, a lipid, a carbohydrate, a polymer, a small molecule, or any combination thereof. Similarly, the methods disclosed herein are not limited by the manner in which the binding target is immobilized onto the solid phase particle, and any techniques that are convenient and compatible with the assay mixture, while retaining in significant degree the binding characteristics of the immobilized binding target are generally contemplated herein. In this fashion, target compounds may be isolated from a mixture by providing binding targets on magnetic beads that correspond to an expected activity of a target compound. Upon binding a bound compound-magnetic particle may be physically manipulated under the influence of a magnetic field and the bound compound may be selectively released upon application of a release agent.

For instance, in certain aspects, the method may include immobilizing the protein to the surface of the solid-phase device by treating Si—OH on the surface of a magnetic particle with aminosilane reagents followed by reaction with glutaraldehyde (GA), the free-end of GA being capable of reacting with the amino groups of lysine to capture a protein binding target, or via streptavidin-biotin interaction or histidine tag. Other mechanisms for immobilizing binding targets onto magnetic particles are known, and contemplated herein as would be understood by those of skill in the art. Magnetic particles and their treatment and handling during HTS methods relying on MS for identification are disclosed herein. Use of other affinity probes in combination with certain aspects disclosed herein is also contemplated as would be understood by those of ordinary skill in the art. Such probes can include a Solid Phase MicroExtraction (SPME) fiber; a REED (as set forth in U.S. Provisional Patent Application No. 62/692,274, the contents of which are incorporated herein); and a magnetic bead.

Forming an assay mixture as it relates to the physical preparation of assay components for methods and systems of the present disclosure is not limited by any particular methodology or machinery, and generally can be any that are suitable to provide and conduct the assay mixture as necessary to screen the selected affinity of a series of compounds. Thus, the formation of the assay mixture can generally comprise the introduction of components explicitly disclosed herein (e.g., a magnetic particle, a plurality of drug candidates, a binding target) as well as any number of additional assay components necessary to conduct the affinity assay. In certain aspects, additional assay components can include assay solvents, buffer salts, stabilizers, and the like. Alternative assay components common to binding assays are also contemplated herein, as would be understood by a person of ordinary skill in the art.

Assay vessels as disclosed herein may be any that are suitable to retain the assay components and allow sampling or transferring assay components to and from the assay mixture in forming the assay mixture and any further operations of the methods disclosed herein. In certain aspects, the assay vessel can comprise a well of a well-plate array having any appropriate dimensions, for instance a 24, 48, 96, 384, or 1536 well plate array. Similarly, the vessel may have any appropriate volume to contain the assay mixture as related to common dimensions of well plates noted above, and thus vessels contemplated herein may have a total volume on the order of 10 μL to 1 mL. Thus, it will be understood that the preparation of an assay mixture as disclosed herein can comprise, in certain aspects, the preparation of a plurality of assay mixtures for serial or parallel analysis, as is commonly performed for high throughput affinity screening assays.

Similarly, introducing assay components can be performed by any method suitable for the assay, particularly high throughput assays. For instance, introduction of the plurality of unique compounds to the assay mixture can comprise serially transferring each of the plurality of compounds from a compound library by liquid dispensers. Liquid dispensing methods (e.g., Mosquito® dispensers) and acoustic dispensers are known for this purpose, and can introduce each of the compounds from a highly concentrated source in nanoliter scale volumes. In this manner, the introduction of thousands of unique compounds into an assay mixture can be prepared in a matter of minutes. It follows then, that, a series of hundreds of assay mixtures such as for a 384 well plate, where each well comprises a nondegenerative combination of comprising unique compounds can be prepared in a matter of hours or days.

Introduction of assay components to the assay vessel can occur in any order, and by any means appropriate to conduct the assay. In certain aspects, introducing assay components can comprise introducing a plurality of unique compounds to the assay vessel, diluting the plurality of compounds with an assay buffer, and introducing the affinity probe to the vessel.

As it relates to the plurality of drug candidates, in certain aspects forming the assay mixture can comprise introducing the plurality of drug candidates into the assay vessel by serial additional of individual compounds from a compound library into an empty well plate. As will be understood by persons of skill in the art, methods of introducing the compounds from a library compound reservoir can be automated such that a well plate containing hundreds of wells each having a combination of 100-2500 unique compounds can be created by selection of a compound library.

Forming an assay mixture also can comprise introducing the magnetic particle into the assay vessel. In certain aspects, the particle may be introduced to the assay vessel independently from other assay components, and prior to the plurality of drug candidates or other assay components. Alternatively, the magnetic particle may be introduced into the assay vessel as a homogenous suspension of magnetic particles within assay buffer, and to complete the assay mixture. Homogenous suspensions of magnetic particles can be achieved by mechanical agitation or by magnetic stirring. As for introducing the plurality of drug candidates into the assay vessel, introducing the magnetic particle can comprise aspirating a homogenous suspension, or by contactless methods such as acoustic transfer. In this manner, assay conditions may be carefully controlled by instantly providing assay conditions in the presence of the plurality of drug candidates, while allowing ample time for the preparation of unique compound plates having many thousands or even millions of drug candidates in a single assay plate.

Forming an assay mixture also can consider the final concentration of individual compounds on the order of μL. Because the concentration of organic solvents such as DMSO typically can be limited to a small percentage to ensure assay compatibility, forming assay mixtures as contemplated herein can comprise diluting the assay mixture using an assay buffer. For instance, where compounds are stored within a compound library as highly concentrated solutions in DMSO (e.g., 10 mM to 10 M), each compound may be transferred in an amount on the order of nanoliters, and diluted with water or an assay buffer to achieve a concentration within each well, with respect to each individual compound added to the well, in a range from 0.1 μM to 100 μM. In this manner, the total concentration of DMSO in the assay mixture can be limited to less than 5%, less than 3%, less than 2% or less than 1%.

Similarly, assay mixtures can be prepared in parallel or serial, or a combination. For instance, each well of well plates can be prepared with a single concentration of hundreds or thousands of compounds within each well. Formation of assay mixtures as contemplated herein can therefore comprise forming a series of assay mixtures within wells of any size well plate, as would be understood by a person of ordinary skill in the art. Thus, in certain aspects, plates comprising 24, 48, 96, 384, or 1536 wells can be prepared such that each well contains an assay mixture prepared as disclosed herein, each well having a combination of hundreds or thousands of compounds to be assayed for affinity.

Once all components of the assay mixture are present, forming the assay mixture can further comprise incubating the plurality of drug candidates and magnetic particle under appropriate assay conditions that allow a binding interaction to form between a set of hit compounds within the plurality of drug candidates and the binding target, e.g., a protein fragment retained on a magnetic particle. Generally, appropriate conditions such as incubation time to reach binding equilibrium, temperature, salt concentration, drug candidate concentration, etc., are dependent on the specific assay, and within ranges understood by those in the art. In certain aspects, incubating the plurality of drug candidates and the magnetic particle can comprise heating the assay mixture to 37° C. for at least 15 minutes prior to preparing the sample for mass spectrometry and mass analysis. In certain aspects, mechanical agitation can be employed in certain aspects to provide a homogenous suspension of the magnetic particle within the assay mixture and ensure that a binding equilibrium is achieved between the binding target and drug candidates within the assay mixture. Mechanical agitation can comprise any method suitable to achieve the desired effect, such as vortexing the assay vessel, coupling a mechanical vibration to the assay vessel, mechanical or magnetic stirring, or by inducing an oscillating magnetic field within the assay mixture as described in detail below. Once a binding interaction between the drug candidates and the magnetic particles reach equilibrium within the assay mixture, the set of hit compounds can be separated from assay components based on the demonstrated binding. Methods for identifying a set of hit compounds from a plurality of drug candidates therefore also can comprise preparatory work up operations following forming the assay mixture. In a general sense, preparing the assay mixture for mass analysis (e.g., sample preparation) as disclosed herein can include separating one or more assay components and the set of hit compounds bound to the magnetic particle. Drug candidates within the assay mixture not bound to the magnetic particle can be separated from the assay mixture, while the hit compound-magnetic particle complex is retained, for instance, by inducing a magnetic field within the assay mixture as described below.

In certain aspects, preparation of the assay mixture for mass analysis also can include disrupting the binding interaction between the set of hit compounds within the plurality of drug candidates.

Magnetic particles contemplated herein generally can comprise any magnetic material, or combination of magnetic and non-magnetic materials, suitable to perform and assist the binding and separation steps contemplated herein. In certain aspects, the magnetic particles can comprise a ferromagnetic particle capable of retaining magnetism without actively applying an external magnetic field, such as by the electromagnets adjacent the assay mixtures as mentioned above. Ferromagnetic particles, including iron and nickel, can be advantageously applied to the physical separation of the hit compounds from the magnetic particles, as the particles will aggregate into larger groups of particles even in the absence of an externally applied magnetic field. Such aggregation can assist in retaining the magnetic particles after disrupting the binding interaction, while the unbound hit compounds are aspirated or ejected from the sample. Ferromagnetic materials appropriate for the magnetic particles contemplated herein include iron and nickel according to methods known by those of ordinary skill in the art. Alternatively, or additionally, magnetic particles contemplated herein can comprise a paramagnetic material that does not retain magnetism without an externally applied magnetic field. Paramagnetic materials can include platinum and tin.

In certain aspects, magnetic particles can comprise a magnetic core coated by a non-magnetic material, the non-magnetic material providing attachment to the binding target. Methods for coating magnetic beads with polymeric surfaces and subsequently treating the surface of the coated particle with a binding target are known in the art. Binding targets can be covalently or non-covalently bound to a surface of the magnetic particle. The size of magnetic particles contemplated herein is not limited to any particular size, and can be any that is convenient to facilitate the transfers and binding steps described herein. Magnetic particles are therefore contemplated herein as being microparticles, nanoparticles or both. Magnetic particles can comprise particles less than 500 μm, less than 100 μm, less than 1 μm, less than 500 nm, less than 100 nm, or less than 10 nm.

In certain aspects, disrupting the binding interaction between the hit compounds and the binding target can comprise separating hit compounds from the binding target by introducing an unbinding solvent into the assay mixture. In certain aspects, separating hit compounds from the binding target can include inserting the magnetic particle and compounds bound to the particle during the assay into an unbinding solvent in a separation vessel to unbind the one or more compounds from the particle. In certain aspects, the resulting mixture in the separation vessel comprising the unbound set of hit compounds, the particle, and the unbinding solvent can be injected into the flowing solvent at the open end of the open port sampling interface.

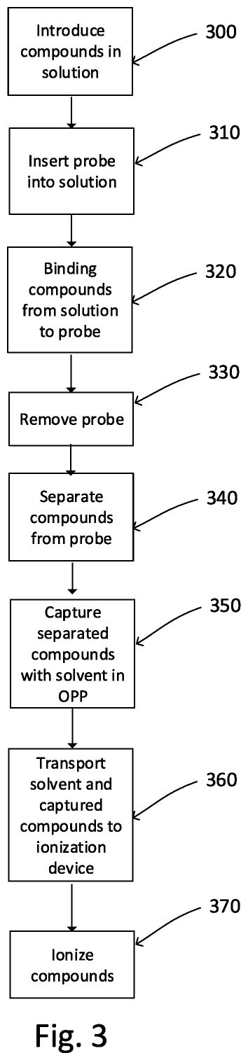

Preparing the assay mixture for mass analysis can comprise any number of operations which can be conducted in any order suitable to provide the set of hit compounds in an appropriate manner for mass analysis. In certain aspects, the hit compound-target complex (i.e. bound compound-magnetic particle component within the solution) can be separated from the assay mixture and washed with an aqueous buffer, prior to separating hit compounds bound to the magnetic particle at the binding target. In another aspect, preparing the assay mixture can include separating hit compounds from magnetic particles within an assay vessel and then introducing the eluted compounds into the open port sampling interface with or without the magnetic particle using a process that does not require the sample to be aspirated off using suction. In aspects where the magnetic particles are retained in position within a sample vessel (and not sampled to the open port interface along with hit compounds) the assay mixture can be aspirated from the assay vessel while retaining the magnetic particle within the assay vessel using a magnetic force positioned inside or adjacent the assay vessel. Wash solution can be added to the magnetic particle, and subsequently removed. Optionally, the magnetic force can be selectively turned off to allow mixing of the bound compound-magnetic particle component with the wash solution prior to turning the magnetic force back on, and aspirating the wash solution. As for other operations involving magnetic particles, mixing of the wash solution can be achieve by mechanical or magnetic means, as disclosed herein for suspending magnetic particles homogenously from other mixtures. Further examples of workflows for preparing the assay mixture are described for several embodiments in the FIGS. 3-10 and below.

In certain aspects, forming the assay mixture and preparing the assay mixture for mass analysis each can independently comprise agitating the assay mixture. For instance, agitation may aid the mixing of assay components while forming the assay mixture to ensure complete dissolution or homogenous suspension. Similarly, reagents may be added to the assay mixture in preparation of separating components of the mixture after forming and conducting the binding assay. For instance, when precipitation aids may be added to assay mixtures in order to separate certain components from the mixture into a solid phase. In aspects where agitation of the assay mixture is desired, agitation can be achieved by mechanical or magnetic stirrers within the assay mixture, sonication probes adjacent to or inserted within the assay mixture, or by applying a mechanical vibration to the assay well itself containing the assay mixture. However, these methods have associated drawbacks including cross-contamination from one assay mixture to another for agitation methods that involve inserting stirrers or probes into the assay mixture, and spilling portions of the assay mixture through applying an external vibration to the assay vessel.

Alternatively, agitation of the assay mixture can be achieved by applying an oscillating magnetic force to the magnetic particles within the assay mixture. The oscillating magnetic force causes the magnetic particles to vibrate and thereby agitates the assay mixture. FIG. 13A demonstrates an exemplar embodiment configured to apply such a magnetic force to the magnetic particles within an assay vessel, e.g., a test tube 1310. As shown in FIG. 13A, a plurality of electromagnets 1320a-1320d can be positioned about the exterior of a test tube. An alternating current then can be applied to the plurality of electromagnets to create an oscillating magnetic field strong enough to couple to magnetic particles 1330 and induce agitation of the assay mixture. In the example of FIG. 13A, the N-S axis of the magnets is in the x-y plane and perpendicular to the vertical axis of a sample well. Alternate arrangements of the electromagnets may be provided, such as aligning the N-S axis of the magnets to be parallel with the vertical axis of a sample well, such as embodiments described in applicant's U.S. Patent Publication No. 2018/0369831, incorporated herein by reference.

The strength of the magnetic field may be adjusted as necessary to achieve adequate coupling of the magnetic force to the magnetic particles, and adequate agitation. In certain aspects, the strength of the magnetic field can be in a range from 1 mT to 1 T, from 10 mT to 500 mT. or from 25 mT to 250 mT. The current and voltage necessary to achieve the appropriate magnetic field strength can vary according to the properties and positioning of the electromagnet with respect to the assay mixture, as will be understood by those of skill in the art. In certain aspects, agitation of the assay mixture can be achieved by applying an alternating current having a frequency of 50 Hz or 60 Hz, or in a range from 10 to 400 Hz, or from 20 to 100 Hz. In certain aspects, each of the frequency and magnitude of the alternating current applied to the electromagnet may be independently variable. In certain aspects, the duration of agitation can be at least 2 seconds, at least 5 seconds, at least 10 seconds, or at least 30 seconds.

The device as described above may also be applied to sequester the magnetic particles from other components of the assay mixture, by inducing a constant magnetic force within the assay mixture. FIG. 13B demonstrates the result of applying a direct current to the electromagnets, and a constant magnetic force within the assay mixture, which moves the magnetic particles 1320 against the edge of test tube 1310. Non-magnetic components of the assay (e.g., solvents and drug candidates not bound to the magnetic particle) remain in the central portion of the assay vessel as supernatant 1340, and can be removed from the assay vessel by any method described herein, for instance, aspiration or acoustic droplet ejection. In certain aspects, the duration of separation can be less than 10 seconds, less than 5 seconds, less than 3 seconds, or less than 1 second.

It is further contemplated that the electromagnets applied as described above and as exemplified by the embodiment shown in FIGS. 13A-13B, allow an assay mixture to be homogenized via agitation and subsequently prepared for mass analysis by sequestering the magnetic particles within the assay mixture. The magnetic force can be oscillated or held constant by applying either alternating or direct current, respectively, to achieve agitation or sequestration of the magnetic particles within the assay mixture. Such aspects can be particularly advantageously combined with acoustic ejection transfer methods as described herein, that can sample a portion of the assay mixture from the center of the assay vessel in a contactless manner. In this sense, the entire assay can be conducted and transferred to the open port interface of a mass spectrometer without introducing extraneous components or machinery into the assay mixture, and without risk of contaminating the assay or subsequent samples.

Sequestering the magnetic particle from the assay mixture can be conducted prior to or after disrupting the binding interaction present between the set of hit compounds and the binding target attached to the magnetic particles. Thus, the process described above of successive agitation and sequestering can be applied to isolate the hit compounds bound to the magnetic particle from other assay components such as solvents, salts, buffers, and other drug candidates present in the assay mixture having no affinity to the binding target. In such aspects, the retained magnetic particles can be reconstituted in a mixture with a wash solution to wash the retained particles. Additionally, a separation agent may be added to disrupt the binding interaction and allowing the set of hit compounds to dissociate from the magnetic particles. The magnetic particles can then be sequestered by applying the constant magnetic field, and the solvated hit compounds can be transferred from the assay mixture without the magnetic particle.

Alternatives to the device embodied by FIG. 13 are also contemplated herein. For instance, the electromagnets of FIG. 13 are positioned in a planar arrangement with respect to walls of the single test tube and assay mixture contained therein. However, certain aspects of devices contemplated herein can comprise electromagnets positioned above the plane of the assay mixture. Such aspects may allow induction of oscillating and constant magnetic fields within assay vessels of various shapes and sizes, for instance the assay well of a well plate as described above. Alternative arrangements of magnetic and electromagnetic assemblies for processing fluids are disclosed in U.S. Publication No. 2020/360879, U.S. Publication No. 2018/0369831, and U.S. Pat. No. 10,656,147, each of which is incorporated herein by reference.

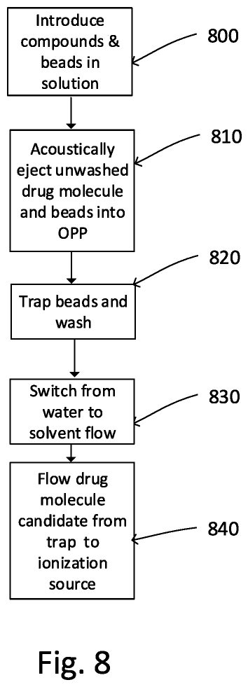

In certain aspects, methods disclosed herein can comprise transferring the sample prior to one or more assay mixture preparatory operations. Indeed, certain aspects can comprise preparing the assay mixture for mass analysis, either partially or completely after transferring a sample containing the set of hit compounds directly to the open port sampling interface of the mass spectrometer. FIG. 8 provides an example of such an aspect, where preparing the assay mixture is conducted within the open port sampling interface by retaining the magnetic particle and flowing a wash solution and an eluent across the magnetic particle to elute the hit compounds into the carrier flow of the mass spectrometer.

Alternatively, preparing the assay mixture can be completed prior to transferring the sample of hit compounds to the open port sampling interface of the mass spectrometer. In such aspects, the assay mixture can be manipulated to provide the set of compounds in an eluent, and at a concentration suitable for mass spectral analysis. In such aspects, it will be appreciated that no special instrumentation is needed within the mass spectrometer and sampling interface, so long as the sampling interface is compatible with the vessel in which the sample containing the hit compounds is prepared. Thus, care can be taken to ensure compatibility is maintained during preparing the assay mixture.

Preparing the assay mixture may be conducted within the assay vessel, within a separate sample vessel, within a transfer conduit, within the open port sampling interface of a mass spectrometer, or any combination thereof. In certain aspects, hit compounds, bound or unbound, can be introduced into the OPI according to a process that filters out the solid phase devices before introduction of ions into the MS. In one embodiment, preparing the assay mixture for mass analysis can be conducted in the assay vessel prior to ejecting the isolated hit compounds and magnetic particle into the OPI where the sample is separated from the magnetic particle using a solvent-based capture fluid. The magnetic particle can then be trapped before entering the MS. Certain aspects can comprise an external magnetic field to trap the solid phase devices before delivering the sample to the MS ion source. In another embodiment, a trap may be provided before the electrospray ionization the OPI or in-line with the transfer conduit.

In certain aspects, hit compounds bound to the magnetic particle can be eluted from the particle prior to transferring a sample of the hit compounds to the MS. For instance, a sample containing the hit compounds can be treated with an organic eluent or an eluent with a high concentration of organic solvents such as methanol and acetonitrile, or combinations and aqueous mixtures thereof. The eluent can be the same or different from the carrier solvent or capture solvent present within the mass spectrometer. Once eluted, the sample containing the set of hit compounds can be sampled directly to the open port sampling interface by any appropriate methodology, including acoustic droplet ejection, flow injection, and automated laboratory systems for introducing nanoliter to microliter amounts of a sample for mass analysis. Acoustic droplet ejection transfers have the additional advantage of being contactless, such as to avoid contamination from one sample to another in high throughput, automated systems. For instance, aspirating a sample from a sample well during transfer can allow some sample to be retained on the aspirating device, and ultimately be transferred to subsequent samples. Acoustic ejection eliminates the possibility of such contamination between samples, as the transfer method is completely contactless between the sample well and the fluid flow of the mass spectrometer via the open-port interface.

In yet a further aspect, preparing the assay mixture for mass analysis may be partially performed in the OPI and/or a transfer conduit, with fewer operations being performed in the assay vessel. For example, a first capture fluid may be used to capture the sample and magnetic particle that provides a washing action as the magnetic particle is trapped with sample, and a second separation fluid (i.e. a solvent) may then be used to separate the sample containing the set of hit compounds from the trapped magnetic particles. In an embodiment, the second separation fluid may flow with a varying concentration gradient where the concentration increases from 0-100% according to a pre-defined ramp or sequence of concentration increases. Also, in an embodiment a MS signal may be used to trigger switching from the first capture fluid to the second separation fluid. In this embodiment, the first capture fluid is directed to the MS, which is useful if the wash components are MS compatible. In another embodiment the capture fluid may be directed to a waste conduit and a timer may be used to trigger switching from the first capture fluid to the second separation fluid and to direct the separation fluid to the ion source, which is useful if the wash components are not MS compatible.

Transferring a sample containing the set of hit compounds also may comprise any particular arrangement of physical transfers and manipulations. For certain aspects, the sample containing the set of hit compounds also can contain the magnetic particle. As an example, the assay mixture can be sampled directly to the open port sampling interface such that the hit compounds are transferred within the assay buffer and bound to the binding target on the magnetic particle. Alternatively, the hit compounds can be eluted from the magnetic particle, but remain in a common suspension without separation prior to transferring the sample to the open port interface. Further still, the hit compound-magnetic particle complex can be separated from aqueous assay components via washing the complex, and the isolated complex then can be transferred to the open port interface.

In aspects where magnetic particles are present within the sample transferred to the open port interface containing the set of hit compounds, it is advantageous that the magnetic particles be reliably separated from the set of hit compounds and removed from the analytical flow prior to ionization in order to prevent damage to the mass spectrometer. Methods are contemplated herein which can further comprise trapping the magnetic particle within a carrier flow of the mass spectrometer, and subsequently releasing the magnetic particle to a waste flow. In this manner, the hit compounds accompanying the magnetic particle can be injected into the analytical flow of the mass spectrometer, while retaining the magnetic particle until such time as can be directed toward a waste flow. Alternatively, the magnetic particles can be subjected to electrospray ionization along with the set of hit compounds, and collected from the exhaust.

Methods disclosed herein may make use of an electromagnet for the purpose of retaining the magnetic particle within the carrier flow until the hit compounds may be separated and entered the carrier flow for analysis. In certain aspects, the magnetic particle may be selectively retained within the open port interface (e.g., by an electromagnet in an operative state) upon transferring the sample containing the set of hit compounds and magnetic particle to the open port. Assay components can be washed from the magnetic particle-hit compound complex to a waste flow, prior to separating the hit compounds from the magnetic particle into a carrier flow while continuing to retain the magnetic particles. The magnetic particles may then be allowed to move through the open port unrestricted toward a waste flow by deactivating the magnetic retention. Certain aspects can comprise switching the flow of the mass spectrometer from a carrier flow to a waste flow to accommodate the embodiment described above. Magnetic particles may also be selectively retained in a similar manner within downstream components of the mass spectrometer prior to ionization.

Separately, the magnetic particles may be allowed to travel within the carrier flow, either bound or unbound to the set of hit compounds (e.g., either with or without separating the compounds), and simply exit the mass analysis flow being discarded during sample vaporization. Such aspects allow for a streamlined process and require no additional mechanisms to achieve the desired outcome, although may not reliably remove magnetic particles from the mass spectrometer.

By these methods described in detail above, it is contemplated that conventional chromatography operations may be removed from the procedure. Liquid chromatography can be employed to achieve separation of hit compounds from the binding target, as described above. Liquid chromatography typically is performed successively on samples and requires a run between 15 minutes and an hour to complete. In highly serial processes such as those described herein, removal of this time-consuming operation can afford significant time savings to the overall screening process.

Successive analyses of assay wells can thus be achieved in quick succession, often on the order of 1 to 10 seconds rather than every 10 minutes or more. Successive analyses therefore approach the sampling limits of acoustic droplet ejection methods for transferring samples to open port sampling interfaces.

Further, reduction in operation time by eliminating chromatography steps can allow a reduction in the number of compounds per assay well. In certain aspects, the methods disclosed herein may be able to conduct high throughput affinity screening wherein the plurality of drug candidates within each assay well is less than 500 compounds, less than 300 compounds, less than 200 compounds, less than 100 compounds, or less than 50 compounds.

Methods disclosed herein also may not be reliant on size-exclusion chromatography as the large magnetic particles with immobilized binding targets are able to be selectively removed from the assay mixture at any number of operations in the methodology without time-intensive chromatography. Embodiments described below illustrate the concepts described and disclosed above.

Certain aspects of the methods disclosed herein can comprise introducing a plurality of drug candidates together in a solution; inserting a particle comprising a surface treatment operative to bind with one or more compounds based on the selected affinity; binding one or more compounds from the plurality of drug candidates to the particle; removing the particle and bound one or more compounds from the solution; separating the one or more compounds from the particle; capturing the separated one or more compounds with flowing solvent at an open end of an open port sampling interface; transporting the solvent and captured one or more compounds to an ionization device; and ionizing the one or more compounds.

In an embodiment, the method may further include analyzing the ionized one or more compounds in a mass spectrometer. In an embodiment, the method may further include, after ionizing the one or more compounds but before the analyzing, separating the ionized one or more compounds based on the difference in high-field and low-field ion mobility provided by a differential mobility spectrometer.

In an embodiment, transferring the sample to the open port interface can comprise injecting or aspirating the unbinding solvent and unbound one or more compounds from the separation vessel and injecting the aspirated unbinding solvent (i.e., eluent) and unbound one or more compounds into a solvent stream pumped to the ionization device. In an embodiment, injecting may include ejecting droplets of the unbinding solvent and unbound one or more compounds from the separation vessel into the flowing solvent at the open end of the open port sampling interface. In an embodiment, transferring the sample can comprise acoustically or pneumatically ejecting droplets of the sample.

In an embodiment, the method may further include sampling the selected drug candidate by acoustically ejecting the selected drug candidate from the assay vessel into the capture fluid within an open port sampling interface. In an embodiment, the method may further include ejecting the selected drug candidate from the assay vessel after washing.

In an embodiment, the method may further include, before the selected drug candidate is ejected from the sample well, separating a bound drug candidate from the magnetic particle, isolating the selected drug candidate from the particle, and ejecting the selected drug candidate without the solid-phase device into the capture fluid of an open port sampling interface. Alternatively, the hit compound (i.e., selected drug candidate) can be ejected in a bound state with the particle. In certain aspects, the selected drug candidate can be eluted from the particle (i.e., unbound) by the capture fluid. Alternatively, the hit compound can be released from the trapped solid-phase device by introducing a wash solution or eluent into the capture fluid. In certain aspects, the eluent can be the same or different than the carrier solvent within the mass spectrometer.

In some embodiments the particle can be transferred to the OPI with the particle separate from the hit compound whereas in other embodiments the particle is transferred with the particle bound to the hit compound as a hit-particle complex.

These together with other aspects and advantages which will be subsequently apparent, reside in the details of construction and operation as more fully hereinafter described and claimed, reference being had to the accompanying drawings forming a part hereof, wherein like numerals refer to like parts throughout. Generally, and as noted above, methods disclosed herein can comprise forming an assay mixture, preparing the assay mixture, and transferring a sample containing the set of hit compounds to an open port sampling interface of a mass spectrometer. As these operations may contain any number of further operations, in any sequential order, specific embodiments are disclosed herein to illustrate certain aspects of the methods disclosed herein. Embodiments provided herein are not intended to limit the scope of the disclosure.

High throughput affinity screening systems are also contemplated, for the purpose of performing methods disclosed herein in a highly automated manner. Generally, the systems can comprise a separate module for performing each operation of the method. In certain aspects, systems contemplated herein can comprise a assay vessel preparation module configured to introduce a plurality of compounds from a compound library into an assay vessel. As above, the plurality of compounds can be any number of compounds, and in certain aspects in a range from 10 to 10,000 compounds, from 500 to 5,000 compounds, or from 1,000 to 2,500 compounds. Systems can comprise an acoustic dispenser variably coupled to compound storage containers to transfer a portion of each selected compound from the container to the assay vessel.

Systems contemplated herein also can comprise an assay module configured to conduct the binding assay on any number of assay vessels within a well plate. In certain aspects, the assay module can comprise magnetic or mechanical agitators, stores of assay components, temperature controls, automated aspirators, and the like for preparing the assay mixture for mass analysis. Assay module may also comprise a magnet (e.g., an electromagnet) attached to a mobile arm, and variably positionable within, or adjacent to, any number assay vessels for retaining the magnetic particle at any point during the assay.

Systems also can comprise an analysis module configured to serially transfer a sample from each well of the well plate into an open port sampling interface of a mass spectrometer and conduct a mass analysis of each sample. In certain aspects the analysis module can comprise an acoustic droplet ejector able to be coupled with any well of the well plate, so as to facilitate serial transfer of samples from a well plate containing sample from the assay module for analysis. Analysis modules may also comprise a magnet (e.g., electromagnet) for selective retaining a magnet particle at any point within the module prior to ionization of the sample. Analysis module can comprise a mass spectrometer, sample vaporization chamber, ionization device, mass fragment detector any additional components necessary to conduct mass spectral analysis. The analysis module may also be configured to automatically correlate mass fragments detected during analysis with those expected from certain compounds within the sample in order to identify the compounds in the sample.

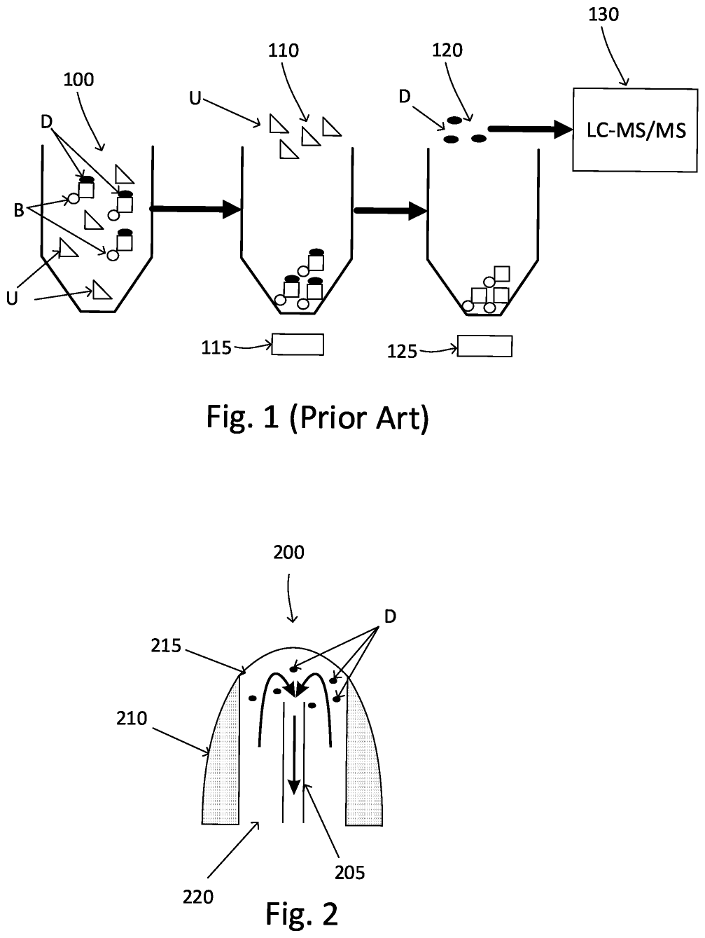

As shown in FIG. 1, the inventors have found that the prior art MagMASS method uses magnetic particles to capture drug molecules with protein binding affinity. First, magnetic beads (B) are introduced to a sample vessel 100 containing drug molecule candidates (U and D) in solution. Drug molecule candidates with affinity (D) then bind to the magnetic beads. The unbound drug molecules (U) are then removed in a wash vessel 110 while the beads (B) and bound drug molecule candidate (D) are retained in the vessel via a magnetic field from magnet 115. The washed beads are removed from the wash vessel and introduced into a separation vessel 120 where the drug molecule candidate (D) is isolated from the beads using a solvent. The isolated drug molecule candidate (d) is then aspirated from the separation vessel 120 while the magnetic beads are held in place via a magnetic field from magnet 125. The aspirated drug molecule candidate is then eluted over time into a LC-MS/MS 130 for analysis. The magnetic beads can then be magnetically removed from the separation vessel 120.

As discussed above, aspects disclosed herein can include an improved method and apparatus for transferring candidate molecules using an OPI with magnetic beads as the solid phase device, and acoustic droplet ejection technology for non-contact introduction of samples to the OPI in a precise and controlled manner.

With reference to FIG. 2, an OPI 200 is shown comprising inner channel 205 as a first cylindrical member disposed within an outer channel 210 as a second cylindrical member arranged in a co-axial arrangement with the inner channel 205, and an open-ended port 215. Additional details of the OPI 200 are provided below with reference to various embodiments.

FIG. 3 discloses an embodiment for identifying and separating drug candidates based on a selected affinity. At 300, a plurality of drug candidates is introduced within the assay vessel as a solution. At 310, a particle is inserted into the solution, where the particle includes a surface treatment operative to bind with one or more compounds based on selected affinity. One or more of the compounds then bind to the particle at 320. In an embodiment, the substrate surface may comprise a Solid Phase Microextraction (SPME) fiber that can contain an embedded protein with binding affinity. The substrate surface may be any material configured to hold the protein and can include various examples such as a mesh material or blade like surface or REED. In other embodiments, as discussed below, the surface treatment can include magnetic material such as beads.

The particle with bound one or more compounds is then removed from the solution at 330. At 340, the one or more compounds are separated from the particle. At 350, the separated one or more compounds are captured with flowing organic solvent at the open-ended port 215 of OPI 200. At 360, the solvent and captured one or more compounds at the open-ended port 215 of OPI 200 are transported to an ionization device, such as MS/MS 130. Then, at 370, the one or more compounds are ionized within MS/MS 130, as is known in the art. In other aspects, chromatography can be excluded prior to MS/MS analysis such that the set of hit compounds is analyzed by MS immediately following preparing the assay mixture.

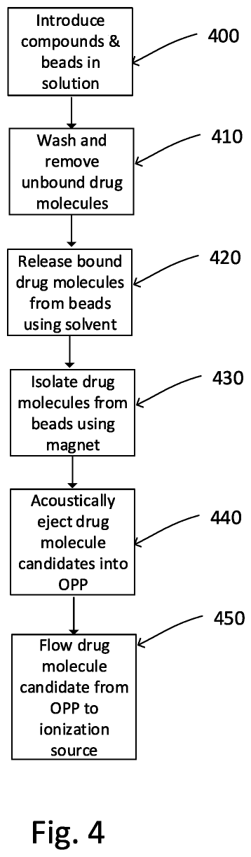

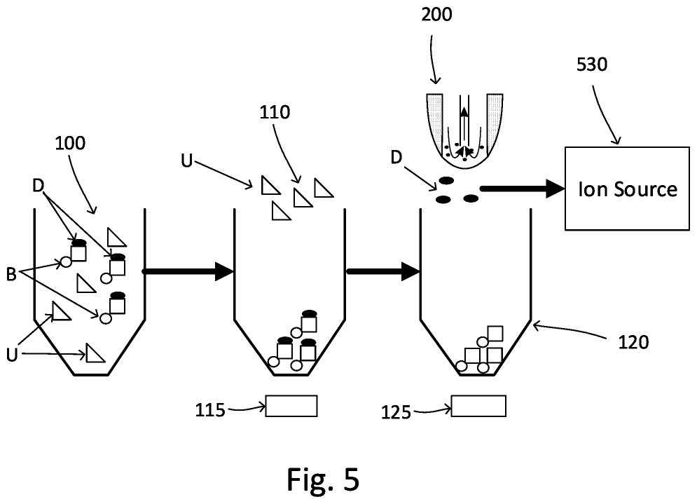

In an embodiment, a method is provided for identifying and separating compounds based on a selected affinity, as set forth in FIG. 4 with reference to the system shown in FIG. 5. At 400, a plurality of drug molecule candidates (U and D) and magnetic beads (B) in solution are introduced to sample vessel 100, for example using an electromagnetic sampling device or probe to which the beads are magnetically attached, such that drug molecule candidates with affinity (D) bind to the magnetic beads. At 410, the beads (B) and bound drug molecule candidates (D) are transferred from the sample vessel 100 to wash vessel 110, for example using the electromagnetic sampling device or probe, whereupon the unbound drug molecules (U) are removed via washing while the beads (B) and bound drug molecule candidates (D) are retained in the vessel via a magnetic field from magnet 115. At 420, the washed beads with bound drug molecule candidates are removed from the wash vessel and introduced into separation vessel 120, for example using the electromagnetic sampling device or probe, where the drug molecule candidates (D) are released from the beads using organic solvent. At 430, the drug molecule candidates (D) are isolated from the magnetic beads (B) via magnet 125. At 440, the drug molecule candidates (D) are acoustically ejected from separation vessel 120 into OPI 200. Within the OPI 200, capture fluid travels towards the tip end 215 through the annular space 220 between the two cylindrical members and then travels away from the tip end through the inner cylinder as depicted in the arrows in the figure defining the fluid path. The capture fluid effectively eliminates the need to clean the sample. At 450, the solvent and ejected drug candidates (D) flow from the tip end 215 to the MS ionization source 530. Optionally or, if necessary, the drug molecule candidate (D) can be separated from the unbound drug molecules (U) using differential mobility spectrometry (DMS) or MS techniques (e.g. fragmentation patterns in MS-MS, etc.)

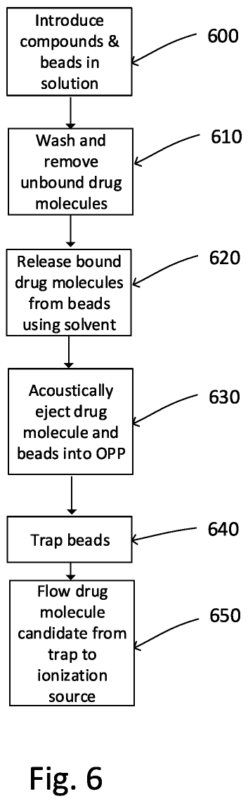

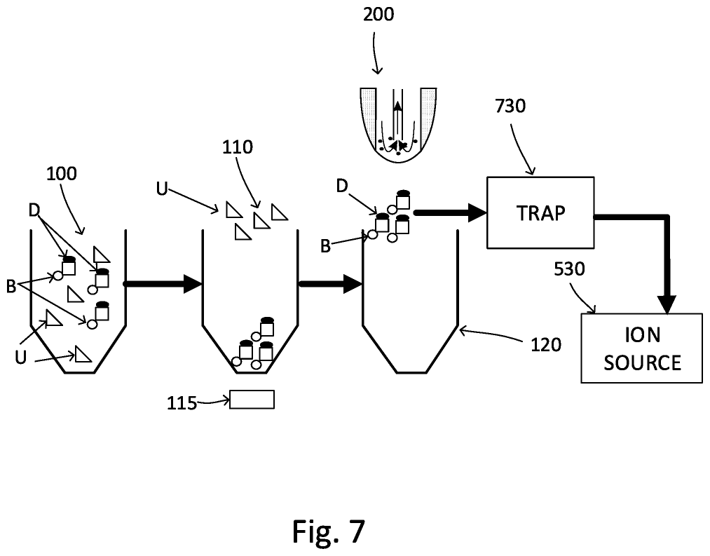

In a further embodiment, a method is provided for identifying and separating compounds based on a selected affinity, as set forth in FIG. 6 with reference to the system shown in FIG. 7. At 600, a plurality of drug molecule candidates (U and D) and magnetic beads (B) in solution are introduced to sample vessel 100, for example using an electromagnetic sampling device or probe to which the beads are magnetically attached, such that drug molecule candidates with affinity (D) bind to the magnetic beads. At 610, the beads (B) and bound drug molecule candidates (D) are transferred from the sample vessel 100 to wash vessel 110, for example using the electromagnetic sampling device or probe, whereupon the unbound drug molecules (U) are removed via washing while the beads (B) and bound drug molecule candidates (D) are retained in the vessel via a magnetic field from magnet 115. At 620, the washed beads with bound drug molecule candidates are removed from the wash vessel and introduced into separation vessel 120, for example using the electromagnetic sampling device or probe, where the drug molecule candidates (D) are released from the beads using organic solvent. At 630, the drug molecule candidates (D) and beads (B) are acoustically ejected from separation vessel 120 into OPI 200. Within the OPI 200, capture fluid travels towards the tip end 215 through the annular space 220 between the two cylindrical members and then travels away from the tip end through the inner cylinder as depicted in the arrows in the figure defining the fluid path. The capture fluid effectively eliminates the need to clean the sample. At 640, the solvent, beads (B) and drug candidates (D) flow from the tip end 215 to an in-line trap 730 where the beads (B) are trapped (640). At 650, the solvent and ejected drug candidates (D) flow from the trap 730 to the MS ionization source 530. Alternatively, rather than separating the drug molecule candidates (D) from the beads in separation vessel 120, the drug molecule candidates (D) may be separated from the beads within OPI 200, where the capture fluid is a solvent operative to release the bond between the drug molecule candidates (D) and the beads.

For acoustic ejection at 630, it is preferable that the drug molecule candidates (D) be uniformly suspended in the sample solution within separation vessel 120, for example by mechanically agitating the separation vessel 120 before dispensing or by integrating an electromagnetic mixer within the acoustic dispensing system.

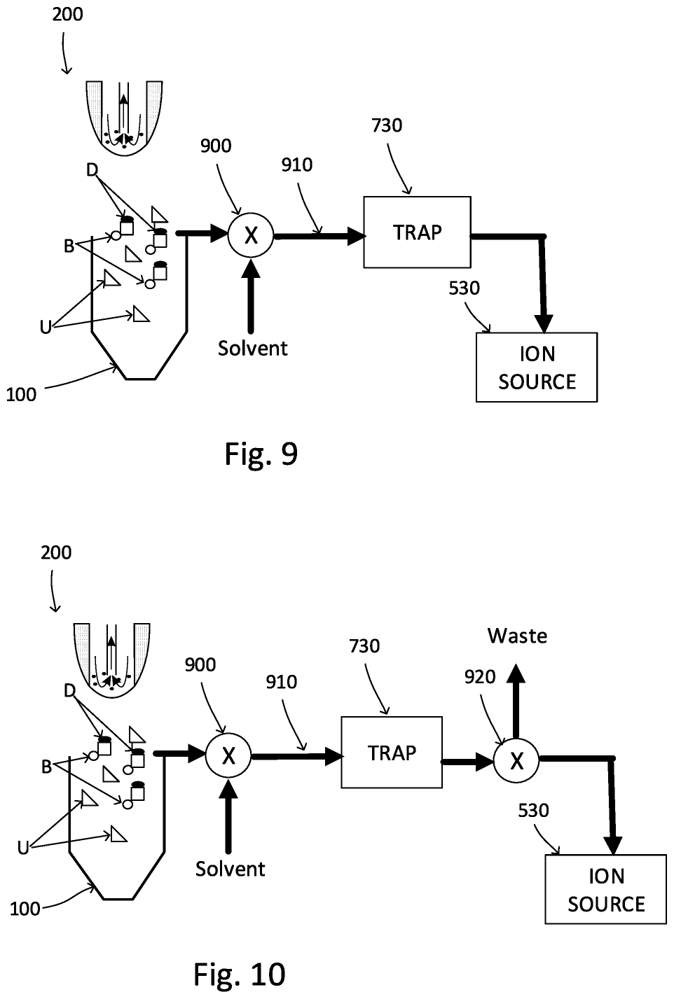

In an additional embodiment, a method is provided for identifying and separating compounds based on a selected affinity, as set forth in FIG. 8 with reference to the system shown in FIG. 9. At 800, a plurality of drug molecule candidates (U and D) and magnetic beads (B) in solution are introduced to sample vessel 100, for example using an electromagnetic sampling device or probe to which the beads are magnetically attached, such that drug molecule candidates with affinity (D) bind to the magnetic beads. At 810, the unwashed drug molecule candidates (D) and beads (B) are acoustically ejected from sample vessel 100 into OPI 200. Within the OPI 200, capture fluid travels towards the tip end 215 through the annular space 220 between the two cylindrical members and then travels away from the tip end through the inner cylinder as depicted in the arrows in the figure defining the fluid path. The capture fluid (e.g. water) effectively eliminates the need to clean the sample. At 820, the solvent, beads (B) and unwashed drug candidates (D) flow from the tip end 215 to an in-line trap 730 where the beads (B) are trapped (640) and the drug candidates (D) are washed to remove unbound drug molecules (U). At 830, the flow of capture fluid (water) is switched to organic solvent flow via a valve 900 to separate the drug molecule candidates (D) from the beads (B). At 840, the solvent and selected drug candidates (D) flow via transport line 910 from the trap 730 to the MS ionization source 530.

Different embodiments of trap 730 are contemplated, including filters or size traps, or a permanent magnet that can be replaced from time to time, or an electromagnet that can be energized to trap magnetic beads (B) and then de-energized, for example during a cleaning cycle, to release any captured magnetic beads. As shown in FIG. 10, the transfer line 900 may include valve(s) 920 to redirect the flow of capture fluid to a waste vessel and thereby avoid releasing magnetic beads into the ionization source 530 during the cleaning cycle, when the electromagnet is de-energized to release captured beads.

In the system of FIG. 7, the trap 730 may be a magnetic trap at the tip end 215 of OPI 200 (i.e. electromagnets surrounding one or both of the first cylindrical member 205 and/or second cylindrical member 210, and wherein a clearing cycle may be performed with a solvent-based capture fluid to release the beads from the trap after the washed drug candidates have been conveyed to the MS ionization source 530.

In another embodiment, the trap 730 may be disposed at the ionization source 530 wherein bead trajectory separates from ions at entrance to the MS ionization source 530 due to the beads being much heavier than the ions, for use with the systems shown in FIGS. 5 and 9.

In a further embodiment, the trap 730 may an in-line magnetic trap on transport line 900 of the system shown in FIG. 9. It is contemplated that the in-line magnetic trap may be a replaceable section of transport line 900 that has a sufficient magnetic field to capture the magnetic beads (B) within the transport line.

It is also contemplated that in the system of FIG. 5, employing acoustic ejection of drug molecule candidates (D) isolated from the beads (B), a permanent magnet guard trap may be included to protect the ionization source 530 and MS form unintentional ejection of magnetic beads from the vessel 120.

Although the systems depicted in FIGS. 5 and 7 discuss the use of separate sample, wash and separation vessels 100, 110 and 120, it is contemplated that sample preparation may be performed in a single vessel or multiple vessels.

In each of the embodiments set forth in FIGS. 4-10, as an alternative to introducing the compounds drug molecules with affinity to the solid phase surfaces of the magnetic particles (B), it is contemplated that the particles (B) can be added after the protein-drug integration in free solution (e.g. after 400, 600, 800), and used to fish-out the protein-drug complex rather than the protein pre-immobilized on magnetic particles (B).

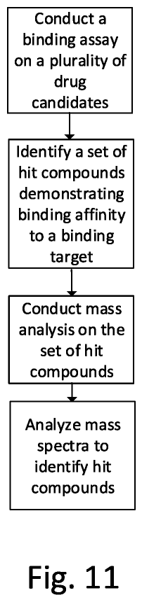

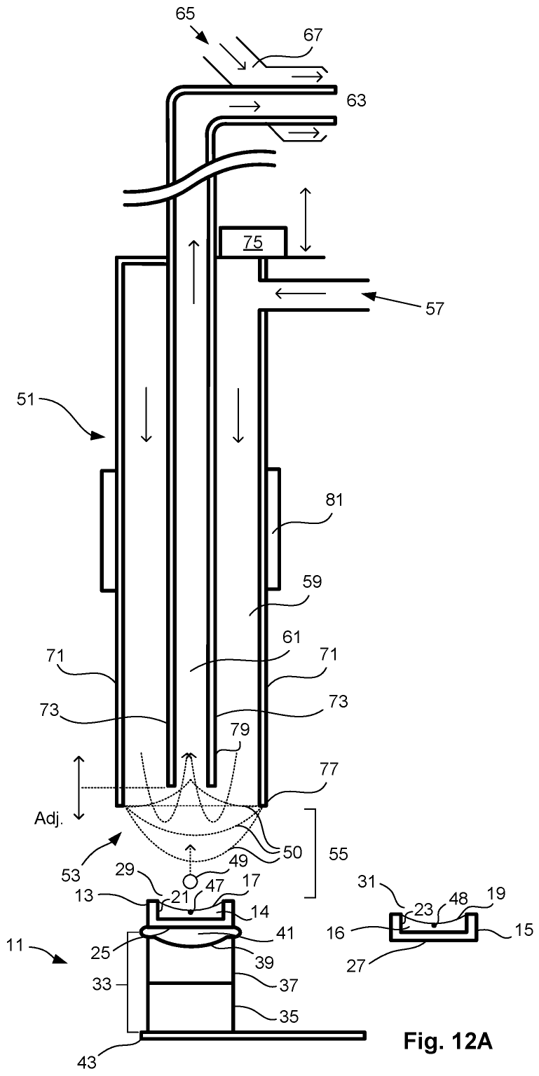

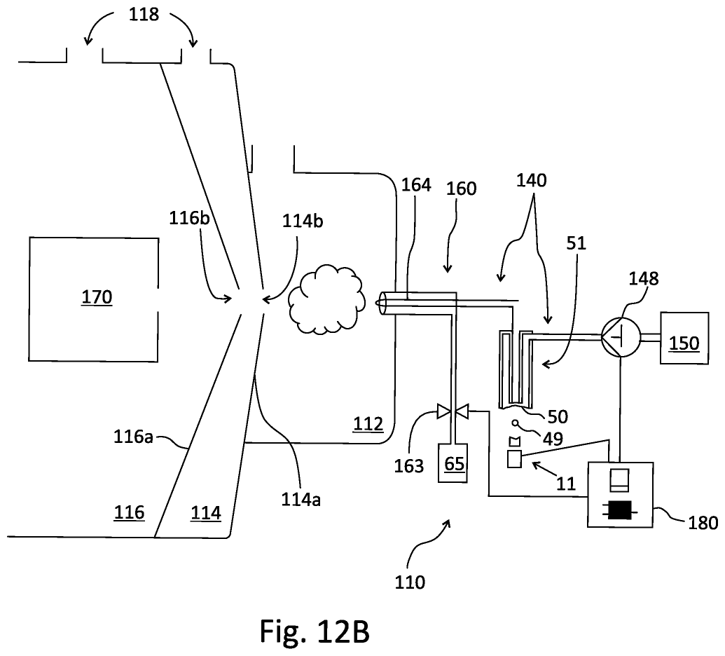

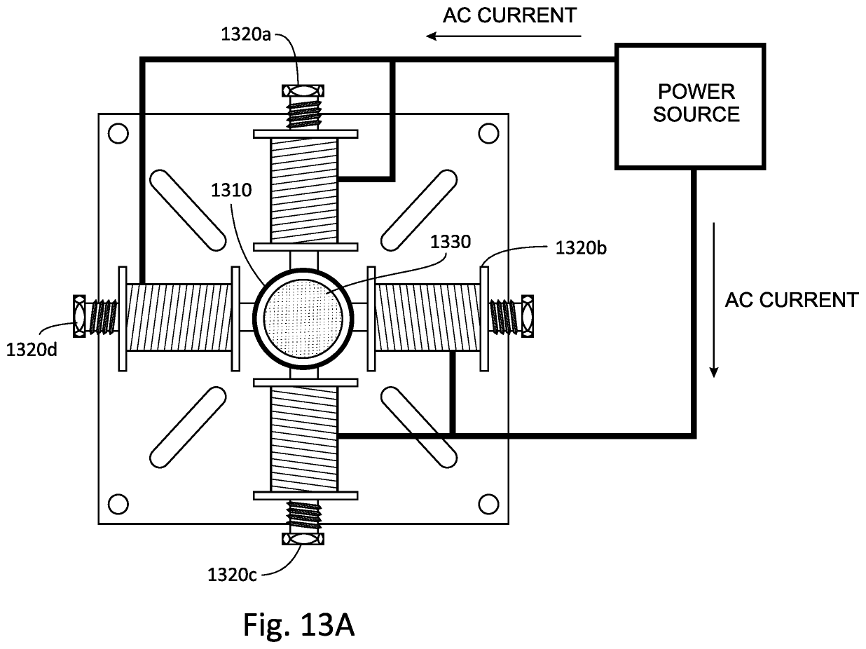

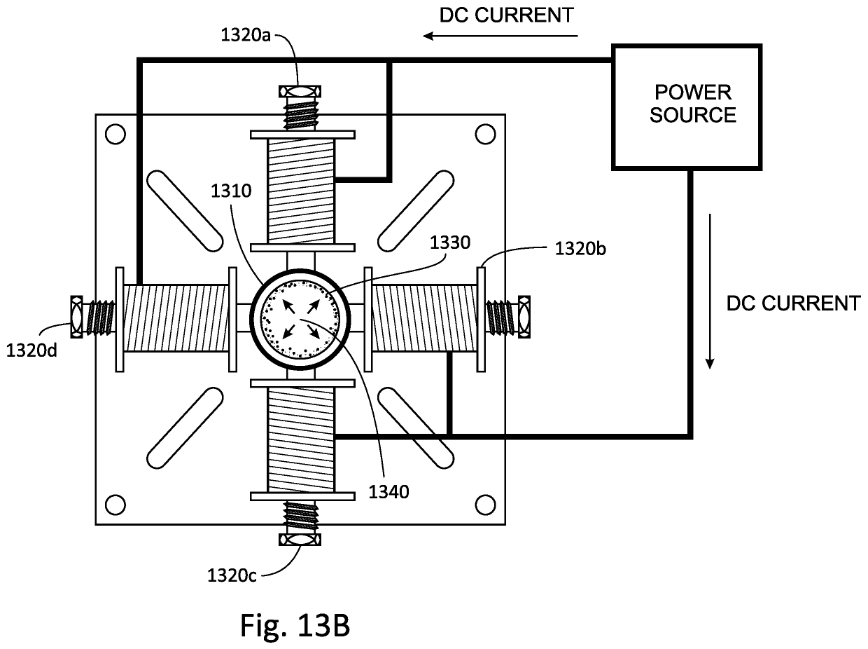

FIG. 11 is provided as a general embodiment similar to that of FIG. 3. The embodiment of FIG. 11 presents provides a method where sequential operations are grouped together in a general manner, and each operation can represent one or more operations performed in any order. Particularly, preparing the assay mixture and transferring the sample are shown as a single operation, where the set of hit compounds is separated from certain assay components and transferred to the mass spectrometer in any of several different arrangements. Thus, it is shown in FIG. 11 that the preparation and transfer of the set of hit compounds identified within the assay can be performed in many different sequential arrangements and locations following the affinity assay and prior to mass analysis, as disclosed herein. Thus, the embodiment shown by FIG. 11 can encompass each of the embodiments disclosed by FIGS. 3-10.

A binding assay may be created by introducing one or more selected compound(s) and magnetic beads into a sample well. The magnetic beads including binding sites for a target compound indicative of a desired binding activity. A assay vessel preparation module may be operative to introduce the selected one or more compound(s) into each sample well of sample plate. An assay module may introduce the magnetic beads that include binding sites corresponding to a desired binding activity of compounds exposed to the magnetic beads. In some embodiments, the assay vessel preparation module and the assay module may comprise separate mechanisms. In some embodiments, the assay vessel preparation module and the assay module may comprise a unified system.

Sample information, corresponding to the one or more compound(s) introduced into that sample well, is associated with the sample well. The sample information may include, for instance, identifier(s) indicative of each of the one or more compounds, reagents, or other information related to analysis of the sample well. The association may be generated by a controller in communication with the assay vessel preparation module and/or the assay module, or may originate from the assay vessel preparation module and/or the assay module and be stored in a memory location accessible by other modules of the system.

The solution in each sample well may be operated on to separate any bound compound-magnetic bead components from the remaining unbound compounds in the solution. For example, the assay module may apply a magnetic force to isolate and retain the bound compound-magnetic bead components within the sample well and transfer out any unbound components in solution. The transfer may occur, for instance by aspiration or other liquid transfer operation such as gravity flow, suction, expiration, or other known means. Alternatively, for example, the assay module may apply a magnetic force to capture and withdraw the bound compound-magnetic bead components from the solution containing any unbound components. In this case, the transfer may occur, for instance by introducing a probe into the solution that is operative to apply a magnetic force to capture the magnetic beads and retain the captured magnetic beads to the probe while the probe is withdrawn from the solution in the sample well.

In either case, the isolated bound compound-bead components are subjected to a washing step that liberates and disposes of any unbound components to produce washed bound compound-magnetic bead components that are free from any unbound components before the bound compound is released from the bead compound and subjected to analysis.

By way of example, a number of different washing steps may be applied, including as described above. If the beads are isolated within the sample well, the assay module may introduce and extract a washing solution to remove any remaining unbound components. If the beads are withdrawn from the sample well, the assay module may transfer the bound compound-magnetic bead components to a washing step that washes any unbound components away before introducing the washed bound compound-magnetic bead components to a clean sample well. The washed bound compound-magnetic bead components may either be separated within a sample well before introduction into an open port interface, or may be ejected from the sample well into the open port interface for separation by the capture liquid flowing through the open port interface.

Regardless of the release mechanism described above, the bound compounds are transferred from the open port interface to the ion source of a mass spectrometer for ionization and subsequent mass analysis by the mass spectrometer. In this manner a microtiter sample well plate may be prepared by the assay vessel preparation module and/or the assay module and compounds introduced to the sample wells that bind to the binding sites of the magnetic beads may be selectively introduced into the mass spectrometer for mass analysis to produce mass analysis results associated with that sample well.

The system as described further provides for identification of compounds that bind to the binding sites of the magnetic beads by an analysis module correlating the mass analysis results from each sample well with the sample information associated with that sample well. The correlation may identify which compounds appear in each mass spectra from the mass analysis results based on the associated sample well information and sample information for that sample well. Accordingly, the system is operative to identify which compound set was introduced into a particular sample well and which compound(s), i.e. bound compounds, were identified by the mass analysis.

A representative system of the invention is illustrated in FIG. 12A. As with all figures referenced herein, in which like parts are referenced by like numerals, FIG. 12A is not to scale, and certain dimensions are exaggerated for clarity of presentation. In FIG. 12A, the acoustic droplet ejection (ADE) device is shown generally at 11, ejecting droplet 49 toward the continuous flow sampling probe (referred to herein as an open port interface (OPI)) indicated generally at 51 and into the sampling tip 53 thereof.

The acoustic droplet ejection device 11 includes at least one reservoir, with a first reservoir shown at 13 and an optional second reservoir 31. In some embodiments a further plurality of reservoirs may be provided. Each reservoir is configured to house a fluid sample having a fluid surface, e.g., a first fluid sample 14 and a second fluid sample 16 having fluid surfaces respectively indicated at 17 and 19. When more than one reservoir is used, as illustrated in FIG. 12A, the reservoirs are preferably both substantially identical and substantially acoustically indistinguishable, although identical construction is not a requirement.

The ADE comprises acoustic ejector 33, which includes acoustic radiation generator 35 and focusing means 37 for focusing the acoustic radiation generated at a focal point 47 within the fluid sample, near the fluid surface. As shown in FIG. 12A, the focusing means 37 may comprise a single solid piece having a concave surface 39 for focusing the acoustic radiation, but the focusing means may be constructed in other ways as discussed below. The acoustic ejector 33 is thus adapted to generate and focus acoustic radiation so as to eject a droplet of fluid from each of the fluid surfaces 17 and 19 when acoustically coupled to reservoirs 13 and 15, and thus to fluids 14 and 16, respectively. The acoustic radiation generator 35 and the focusing means 37 may function as a single unit controlled by a single controller, or they may be independently controlled, depending on the desired performance of the device.

The acoustic droplet ejector 33 may be in either direct contact or indirect contact with the external surface of each reservoir. With direct contact, in order to acoustically couple the ejector to a reservoir, it is preferred that the direct contact be wholly conformal to ensure efficient acoustic energy transfer. That is, the ejector and the reservoir should have corresponding surfaces adapted for mating contact. Thus, if acoustic coupling is achieved between the ejector and reservoir through the focusing means, it is desirable for the reservoir to have an outside surface that corresponds to the surface profile of the focusing means. Without conformal contact, efficiency and accuracy of acoustic energy transfer may be compromised. In addition, since many focusing means have a curved surface, the direct contact approach may necessitate the use of reservoirs that have a specially formed inverse surface.

Optimally, acoustic coupling is achieved between the ejector and each of the reservoirs through indirect contact, as illustrated in FIG. 12A. In the figure, an acoustic coupling medium 41 is placed between the ejector 33 and the base 25 of reservoir 13, with the ejector and reservoir located at a predetermined distance from each other. The acoustic coupling medium may be an acoustic coupling fluid, preferably an acoustically homogeneous material in conformal contact with both the acoustic focusing means 37 and the underside of the reservoir. In addition, it is important to ensure that the fluid medium is substantially free of material having different acoustic properties than the fluid medium itself. As shown, the first reservoir 13 is acoustically coupled to the acoustic focusing means 37 such that an acoustic wave generated by the acoustic radiation generator is directed by the focusing means 37 into the acoustic coupling medium 41, which then transmits the acoustic radiation into the reservoir 13.

In operation, reservoir 13 and optional reservoir 15 of the device are filled with first and second fluid samples 14 and 16, respectively, as shown in FIG. 12A. The acoustic ejector 33 is positioned just below reservoir 13, with acoustic coupling between the ejector and the reservoir provided by means of acoustic coupling medium 41. Initially, the acoustic ejector is positioned directly below sampling tip 53 of OPI 51, such that the sampling tip faces the surface 17 of the fluid sample 14 in the reservoir 13. Once the ejector 33 and reservoir 13 are in proper alignment below sampling tip 53, the acoustic radiation generator 35 is activated to produce acoustic radiation that is directed by the focusing means 37 to a focal point 47 near the fluid surface 17 of the first reservoir. As a result, droplet 49 is ejected from the fluid surface 17 toward and into the liquid boundary 50 at the sampling tip 53 of the OPI 51, where it combines with solvent in the flow probe 53.

The profile of the liquid boundary 50 at the sampling tip 53 may vary from extending beyond the sampling tip 53 to projecting inward into the OPI 51, as described in more detail below in relation to FIG. 2. In a multiple-reservoir system, the reservoir unit (not shown), e.g., a multi-well plate or tube rack, can then be repositioned relative to the acoustic ejector such that another reservoir is brought into alignment with the ejector and a droplet of the next fluid sample can be ejected. The solvent in the flow probe cycles through the probe continuously, minimizing or even eliminating “carryover” between droplet ejection events. Fluid samples 14 and 16 are samples of any fluid for which transfer to an analytical instrument is desired, where the term “fluid” is as defined earlier herein.

The structure of OPI 51 is also shown in FIG. 12A. Any number of commercially available continuous flow sampling probes can be used as is or in modified form, all of which, as is well known in the art, operate according to substantially the same principles. As can be seen in the FIG. 12A, the sampling tip 53 of OPI 51 is spaced apart from the fluid surface 17 in the reservoir 13, with a gap 55 therebetween. The gap 55 may be an air gap, or a gap of an inert gas, or it may comprise some other gaseous material; there is no liquid bridge connecting the sampling tip 53 to the fluid 14 in the reservoir 13. The OPI 51 includes a solvent inlet 57 for receiving solvent from a solvent source and a solvent transport capillary 59 for transporting the solvent flow from the solvent inlet 57 to the sampling tip 53, where the ejected droplet 49 of analyte-containing fluid sample 14 combines with the solvent to form an analyte-solvent dilution. A solvent pump (not shown) is operably connected to and in fluid communication with solvent inlet 57 in order to control the rate of solvent flow into the solvent transport capillary and thus the rate of solvent flow within the solvent transport capillary 59 as well.

Fluid flow within the OPI 51 carries the analyte-solvent dilution through a sample transport capillary 61 provided by inner capillary tube 73 toward sample outlet 63 for subsequent transfer to an analytical instrument. A sampling pump (not shown) can be provided that is operably connected to and in fluid communication with the sample transport capillary 61, to control the output rate from outlet 63. In a preferred embodiment, a positive displacement pump is used as the solvent pump, e.g., a peristaltic pump, and, instead of a sampling pump, an aspirating nebulization system is used so that the analyte-solvent dilution is drawn out of the sample outlet 63 by the Venturi effect caused by the flow of the nebulizing gas introduced from a nebulizing gas source 65 via gas inlet 67 (shown in simplified form in FIG. 12A, insofar as the features of aspirating nebulizers are well known in the art) as it flows over the outside of the sample outlet 63. The analyte-solvent dilution flow is then drawn upward through the sample transport capillary 61 by the pressure drop generated as the nebulizing gas passes over the sample outlet 63 and combines with the fluid exiting the sample transport capillary 61. A gas pressure regulator is used to control the rate of gas flow into the system via gas inlet 67. In a preferred manner, the nebulizing gas flows over the outside of the sample transport capillary 61 at or near the sample outlet 63 in a sheath flow type manner which draws the analyte-solvent dilution through the sample transport capillary 61 as it flows across the sample outlet 63 that causes aspiration at the sample outlet upon mixing with the nebulizer gas.

The solvent transport capillary 59 and sample transport capillary 61 are provided by outer capillary tube 71 and inner capillary tube 73 substantially co-axially disposed therein, where the inner capillary tube 73 defines the sample transport capillary, and the annular space between the inner capillary tube 73 and outer capillary tube 71 defines the solvent transport capillary 59.

The system can also include an adjuster 75 coupled to the outer capillary tube 71 and the inner capillary tube 73. The adjuster 75 can be adapted for moving the outer capillary tube tip 77 and the inner capillary tube tip 79 longitudinally relative to one another. The adjuster 75 can be any device capable of moving the outer capillary tube 71 relative to the inner capillary tube 73. Exemplary adjusters 75 can be motors including, but are not limited to, electric motors (e.g., AC motors, DC motors, electrostatic motors, servo motors, etc.), hydraulic motors, pneumatic motors, translational stages, and combinations thereof. As used herein, “longitudinally” refers to an axis that runs the length of the probe 51, and the inner and outer capillary tubes 73, 71 can be arranged coaxially around a longitudinal axis of the probe 51, as shown in FIG. 1.

Additionally, as illustrated in FIG. 12A, the OPI 51 may be generally affixed within an approximately cylindrical holder 81, for stability and ease of handling.

FIG. 12B schematically depicts an embodiment of an exemplary system 110 in accordance with various aspects of the applicant's teachings for ionizing and mass analyzing analytes received within an open end of a sampling probe 51, the system 110 including an acoustic droplet injection device 11 configured to inject a droplet 49, from a reservoir into the open end of the sampling probe 51. As shown in FIG. 12B, the exemplary system 110 generally includes a sampling probe 51 (e.g., an open port probe) in fluid communication with a nebulizer-assisted ion source 160 for discharging a liquid containing one or more sample analytes (e.g., via electrospray electrode 164) into an ionization chamber 112, and a mass analyzer 170 in fluid communication with the ionization chamber 112 for downstream processing and/or detection of ions generated by the ion source 160. A fluid handling system 140 (e.g., including one or more pumps 143 and one or more conduits) provides for the flow of liquid from a solvent reservoir 150 to the sampling probe 51 and from the sampling probe 51 to the ion source 160. For example, as shown in FIG. 12B, the solvent reservoir 150 (e.g., containing a liquid, desorption solvent) can be fluidly coupled to the sampling probe 51 via a supply conduit through which the liquid can be delivered at a selected volumetric rate by the pump 143 (e.g., a reciprocating pump, a positive displacement pump such as a rotary, gear, plunger, piston, peristaltic, diaphragm pump, or other pump such as a gravity, impulse, pneumatic, electrokinetic, and centrifugal pump), all by way of non-limiting example. As discussed in detail below, flow of liquid into and out of the sampling probe 51 occurs within a sample space accessible at the open end such that one or more droplets can be introduced into the liquid boundary 50 at the sample tip 53 and subsequently delivered to the ion source 160. As shown, the system 110 includes an acoustic droplet injection device 11 that is configured to generate acoustic energy that is applied to a liquid contained with a reservoir (as depicted in FIG. 12A) that causes one or more droplets 49 to be ejected from the reservoir into the open end of the sampling probe 51. A controller 180 can be operatively coupled to the acoustic droplet injection device 11 and can be configured to operate any aspect of the acoustic droplet injection device 11 (e.g., focusing means, acoustic radiation generator, automation means for positioning one or more reservoirs into alignment with the acoustic radiation generator, etc.) so as to inject droplets into the sampling probe 51 or otherwise discussed herein substantially continuously or for selected portions of an experimental protocol by way of non-limiting example.

As shown in FIG. 12B, the exemplary ion source 160 can include a source 65 of pressurized gas (e.g. nitrogen, air, or a noble gas) that supplies a high velocity nebulizing gas flow which surrounds the outlet end of the electrospray electrode 164 and interacts with the fluid discharged therefrom to enhance the formation of the sample plume and the ion release within the plume for sampling by 114b and 116b, e.g., via the interaction of the high speed nebulizing flow and jet of liquid sample (e.g., analyte-solvent dilution). The nebulizer gas can be supplied at a variety of flow rates, for example, in a range from about 0.1 L/min to about 20 L/min, which can also be controlled under the influence of controller 180 (e.g., via opening and/or closing valve 163). In accordance with various aspects of the present teachings, it will be appreciated that the flow rate of the nebulizer gas can be adjusted (e.g., under the influence of controller 180) such that the flow rate of liquid within the sampling probe 51 can be adjusted based, for example, on suction/aspiration force generated by the interaction of the nebulizer gas and the analyte-solvent dilution as it is being discharged from the electrospray electrode 164 (e.g., due to the Venturi effect).

In the depicted embodiment, the ionization chamber 112 can be maintained at an atmospheric pressure, though in some embodiments, the ionization chamber 112 can be evacuated to a pressure lower than atmospheric pressure. The ionization chamber 112, within which the analyte can be ionized as the analyte-solvent dilution is discharged from the electrospray electrode 164, is separated from a gas curtain chamber 114 by a plate 114a having a curtain plate aperture 114b. As shown, a vacuum chamber 116, which houses the mass analyzer 170, is separated from the curtain chamber 114 by a plate 116a having a vacuum chamber sampling orifice 116b. The curtain chamber 114 and vacuum chamber 116 can be maintained at a selected pressure(s) (e.g., the same or different sub-atmospheric pressures, a pressure lower than the ionization chamber) by evacuation through one or more vacuum pump ports 118.

It will also be appreciated by a person skilled in the art and in light of the teachings herein that the mass analyzer 170 can have a variety of configurations. Generally, the mass analyzer 170 is configured to process (e.g., filter, sort, dissociate, detect, etc.) sample ions generated by the ion source 160. By way of non-limiting example, the mass analyzer 170 can be a triple quadrupole mass spectrometer, or any other mass analyzer known in the art and modified in accordance with the teachings herein. Other non-limiting, exemplary mass spectrometer systems that can be modified in accordance various aspects of the systems, devices, and methods disclosed herein can be found, for example, in an article entitled “Product ion scanning using a Q-q-Qₗᵢₙₑₐᵣ ion trap (Q TRAP®) mass spectrometer,” authored by James W. Hager and J. C. Yves Le Blanc and published in Rapid Communications in Mass Spectrometry (2003; 17: 1056-1064), and U.S. Pat. No. 7,923,681, entitled “Collision Cell for Mass Spectrometer,” which are hereby incorporated by reference in their entireties. Other configurations, including but not limited to those described herein and others known to those skilled in the art, can also be utilized in conjunction with the systems, devices, and methods disclosed herein. For instance, other suitable mass spectrometers include single quadrupole, triple quadrupole, ToF, trap, and hybrid analyzers. It will further be appreciated that any number of additional elements can be included in the system 110 including, for example, an ion mobility spectrometer (e.g., a differential mobility spectrometer) that is disposed between the ionization chamber 112 and the mass analyzer 170 and is configured to separate ions based on their mobility through a drift gas in high- and low-fields rather than their mass-to-charge ratio). Additionally, it will be appreciated that the mass analyzer 170 can comprise a detector that can detect the ions which pass through the analyzer 170 and can, for example, supply a signal indicative of the number of ions per second that are detected.

1. A method for identifying a set of hit compounds having a selected affinity to a binding target, the method comprising:
forming an assay mixture within an assay vessel, the assay mixture comprising a plurality of drug candidates and at least one binding target immobilized onto a plurality of magnetic particles;
preparing at least a portion of the assay mixture for mass analysis; and
transferring a sample of the assay mixture containing the set of hit compounds to an open port sampling interface of a mass spectrometer.

2. The method of claim 1, wherein forming the assay mixture comprises:
introducing the plurality of drug candidates into the assay vessel by serially adding individual compounds from a compound library;
introducing the magnetic particles into the assay vessel; and
incubating the plurality of drug candidates and the magnetic particles under assay conditions.

3. The method of claim 1, wherein preparing at least a portion of the assay mixture comprises:
separating one or more components of the assay mixture from the set of hit compounds; and
disrupting a binding interaction between the set of hit compounds and the binding target.

4. The method of claim 1, wherein transferring the sample is conducted partially or completely prior to preparing the assay mixture for mass analysis.

5. The method of claim 1, wherein the sample containing the set of hit compounds comprises bound compound-magnetic particle components within the assay mixture.

6. The method of claim 1, further comprising trapping the magnetic particles within a carrier flow of the mass spectrometer by selectively activating a magnetic force, and subsequently releasing the magnetic particles to a waste flow by selectively deactivating the magnetic force.

7. The method of claim 6, comprising trapping the magnetic particle within the open port sampling interface or within a downstream component of the open port sampling interface prior to ionization.

8. The method of claim 5, wherein the magnetic particle is discarded via exhaust from a sample vaporization chamber of the mass spectrometer.

9. The method of claim 1, wherein transferring a sample containing the set of hit compounds comprises transferring a sample of the assay mixture directly from the assay vessel to the open port sampling interface.

10. The method of claim 3, wherein preparing the assay mixture consists of:
inserting a magnet into the assay mixture to retain the magnetic particles adjacent to the magnet;
removing the magnet and retained magnetic particle from the assay mixture;
optionally washing the magnetic particles retained adjacent to the magnet with a wash solution; and
contacting the magnetic particle with a release agent while the magnetic particles are retained adjacent to the magnet, thereby separating hit compounds from the binding target and forming the sample containing a set of hit compounds to be transferred to the open port sampling interface.

11. The method of claim 3, wherein disrupting the binding interaction comprises introducing an unbinding solvent to the assay mixture to separate the set of hit compounds from the binding target.

12. The method of claim 1, further comprising:
applying an oscillating magnetic force to the magnetic particle to agitate the assay mixture;
applying a constant magnetic force to the magnetic particles to retain magnetic particles at a position within the assay mixture; or
both.

13. The method of claim 3, wherein preparing the assay mixture consists of:
applying a magnetic force adjacent the assay vessel to retain the magnetic particles within the assay vessel;
aspirating at least a portion of the assay mixture from the assay vessel;
optionally washing the magnetic particles within the assay vessel; and
adding a separation agent to the assay vessel to separate hit compounds from the binding target.

14. (canceled)

15. The method of claim 1, further comprising analyzing the set of hit compounds by mass spectrometry, without liquid chromatography.

16. The method of claim 1, wherein forming an assay mixture comprises introducing magnetic particles into the assay vessel, each magnetic particle including at least one binding site for binding to at least one target compound.

17. The method of claim 1, further comprising generating sample information for the assay mixture, wherein the sample information includes an identifier indicative of each of the one or more compounds, reagents, or other information related to analysis of the sample well.

18-20. (canceled)

21. An automated high throughput screening system, the system comprising:
a assay vessel preparation module configured to introduce a plurality of compounds from a compound library into an assay vessel;
an assay module configured to conduct a binding assay comprising introducing magnetic particles into the assay vessel, each magnetic particle including at least one binding site for binding to at least one target compound; and
an analysis module configured to serially transfer a sample from the assay vessel into an open port sampling interface of a mass spectrometer and conduct a mass analysis of the transferred sample.

22-23. (canceled)

24. The system of claim 21, further comprising sample information associated with the assay vessel indicative of the plurality of compounds introduced into the assay vessel, wherein the analysis module correlates the sample information with the mass analysis generated from the assay vessel to identify any hit compounds that bound to the magnetic particles.

25. The system of claim 21, further comprising
a magnetic trap positioned in fluid communication with the open port sampling interface and situated to trap magnetic particles during transfer from the open sampling interface before introduction to an inlet of the mass spectrometer.

26. The system of claim 21, further comprising an identifier associated with the assay vessel and sample information corresponding to the plurality of compounds introduced into the assay vessel.

27. (canceled)
